(19) United States
(12) Plant Patent Application Publication
(10) Pub. No.: US 2005/0251889 P1
(43) Pub. Date: Nov. 10, 2005

(54) Chrysanthemum plant named 'Redock Yellow'

(75) Inventor: Mark Roland Boeder, The Hague (NL)

Correspondence Address: Charles A. Wendel;Steptoe and Johnson LLP, 1330 Connecticut Ave N.W., Washington, DC, 20036, US

(73) Assignee: Chrysanthemum Breeders Association, N.V., Aalsmeer (NL)

(21) Appl. No.: 10/841,483

(22) Filed: May 10, 2004

(51) Int. Cl. A01H 5/00

(52) U.S. Cl. PLT/295

(57) ABSTRACT

A Chrysanthemum plant named ‘Redock Yellow’ characterized by its medium sized single type blooms with yellow ray-florets and yellow-green disc florets.

### RELATED CULTIVARS

‘Redock Yellow’ is related to ‘Redock White’ (copending U.S. Plant patent application), ‘Redock Dark’ (U.S. Plant Pat. pending), ‘Redock Salmon’ (U.S. Plant Pat. pending), and ‘Redock Orange’ (U.S. Plant Pat. pending). These varieties are all mutants of the original ‘Redock’ (unpatented).

### BACKGROUND OF THE INVENTION

‘Redock Yellow’ is a product of a breeding and selection program which had the objective of finding color mutants of ‘Redock’. The new plant of the present invention comprises a new and distinct cultivar of chrysanthemum plant that is a natural occurring sport of a parent chrysanthemum named ‘Redock’ (unpatented). A comparison with Parent chrysanthemum ‘Redock’ is also made in this application. The new cultivar was discovered as a whole plant mutation in 2001 by Mark Roland Boeder in a controlled environment (greenhouse) in Rijsenhout Holland. The first act of asexual reproduction of ‘Redock Yellow’ was accomplished when vegetative cuttings were taken from the initial selection in 2001 in Rijsenhout Holland.

### SUMMARY OF THE INVENTION

The present invention is a new and distinct variety of chrysanthemum bearing medium sized blooms with yellow ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

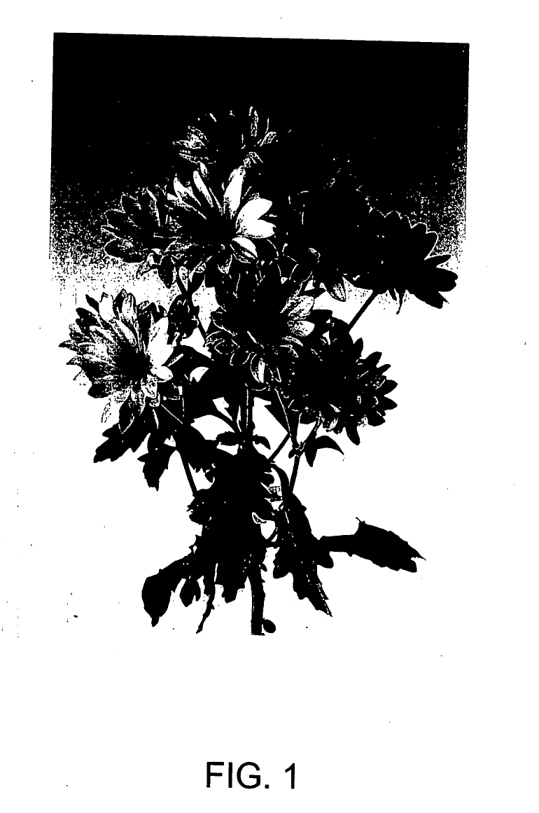

FIG. 2 shows the bloom the cultivar in various stages

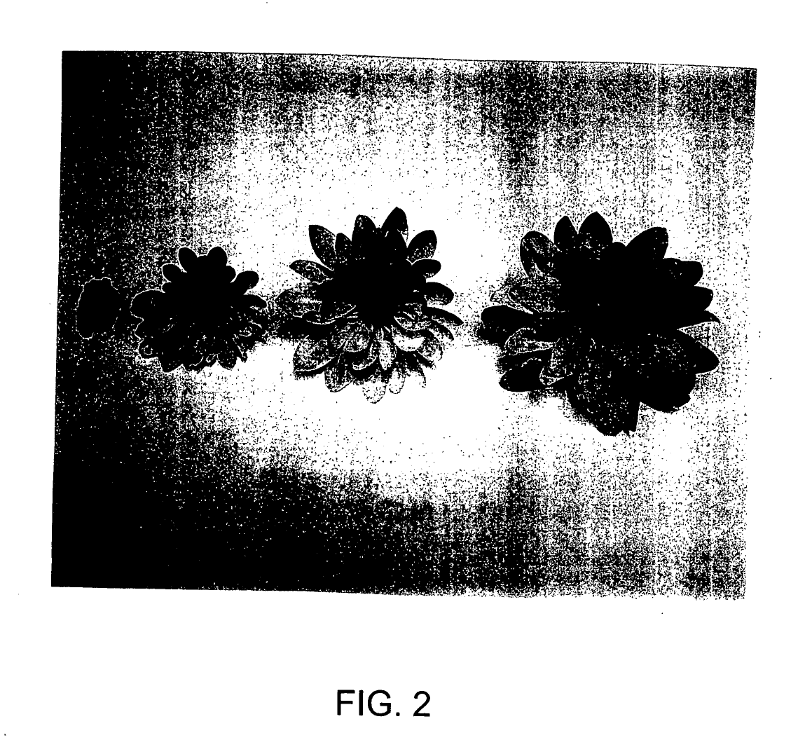

FIG. 3 shows the foliage of the cultivar

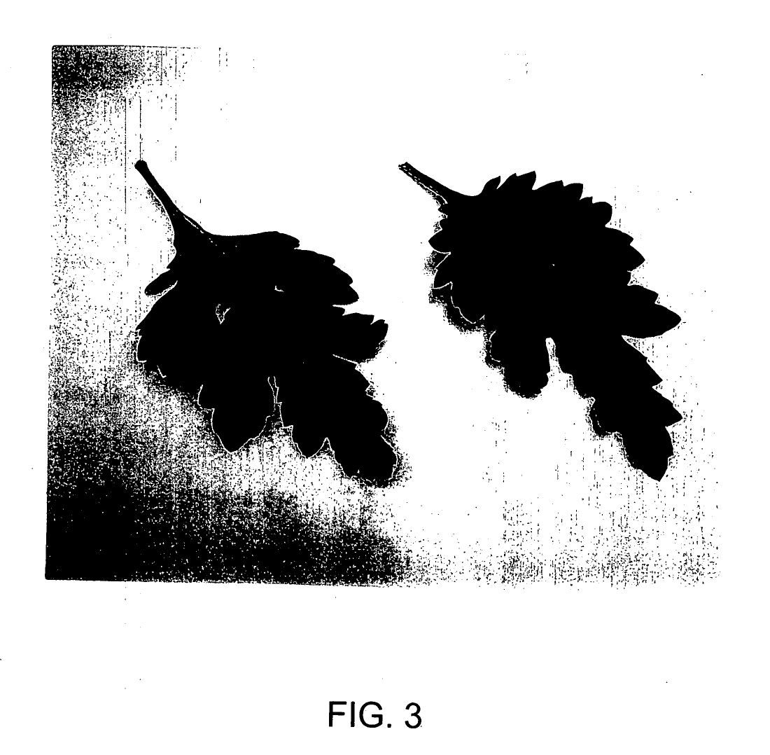

### DESCRIPTION OF THE INVENTION

This new variety of chrysanthemum is of the botanical classification Chrysanthemum morifolium L. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 47 days. After the long day period to flowering growth retardants were applied 3 times in an average dose of 2.5 gram/liter water. No tests were done on disease or insect resistance or susceptibility. This new variety produces medium sized blooms with yellow ray-florets and yellow-green disc-florets blooming on the plant for 4 weeks. This new variety of chrysanthemum has been found to retain its distinctive characteristics throughout successive propagations however the phenotype may vary significantly with variations in environment such as light intensity and temperature. To show the phenotype as described ‘Redock Yellow’ can be planted without assimilation lightning (high pressure sodium lamps) between week 1 and week 52 under greenhouse conditions in Holland. With assimilation light (minimum level 2500 lux) it can be planted year round under greenhouse conditions in Holland. The difference between ‘Redock’ and its sport ‘Redock Yellow’ is flower color. From the cultivars known to inventor the most similar existing cultivars in comparison to ‘Redock Yellow’ are the original ‘Redock’ and its several sports and ‘Yellow Reagan’ (U.S. Plant Pat. No. 8,884). When ‘Redock Yellow’ is being compared with ‘Redock’, the following differences and similarities are noticed: The difference of ‘Redock Yellow’, and ‘Redock’ is the (I) ray-floret color. The color of ‘Redock Yellow’ is yellow, that of ‘Redock’ is pink. All other characteristics of members of the ‘Redock’ family are similar. While when ‘Redock Yellow’ is being compared with ‘Yellow Reagan’, the following differences and similarities are noticed. (1) ray-floret color. Both ‘Redock Yellow’ and ‘Yellow Reagan’ have yellow florets. (2) height of bloom. The bloom height of ‘Redock Yellow’ is high, while that of ‘Yellow Reagan’ is low. (3) cross-section of ray-florets. This is strongly concave for ‘Redock Yellow’, while it is flat for ‘Yellow Reagan’. (4) vigour. The plants of ‘Redock Yellow’ are more vigorous than those of ‘Yellow Reagan’. (5) plant height. The plants of ‘Redock Yellow’ are higher than ‘Yellow Reagan’. The following is a description of the plant and characteristics that distinguish ‘Redock Yellow’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of
CULTIVAR ‘Redock Yellow’

| | |
|---|---|
| Bud | |
| Size | Small, cross-section 0.7 cm |
| | height 0.9 cm |
| Outside Color | Yellow-green 150D |
| Involucral bracts | 3 rows, length 8 mm, width 3 mm |
| Involucral bracts among disc-florets | Not present |
| Involucral bracts color | Green 147 B |
| Bloom | |
| Type | Single |
| Height | High |
| Size | Medium |
| Fully Expanded | 7.0 cm |
| Number of blooms per single stem | Average of 15-20 |
| Seeds | Not produced |
| Performance (blooming period) | 4 weeks |
| Fragrance | Typical chrysanthemum |
| Color | |
| Center of the flower (disc-florets) | Immature yellow-green 144 A |
| | Mature yellow-green 151 C |
| Color of upper surface of the majority | Yellow 7B |
| of the ray-florets | |
| Color of the lower surface of the | Yellow 7C |
| majority of the ray-florets | |
| Tonality from Distance | A spray mum with white flowers |
| | and a yellow-green disc |
| Color of the upper surface of the | Yellow 7B |
| ray-florets after aging of the plant | |
| Ray florets | |
| Texture | Upper and under side smooth |
| Number | 26-28 |
| Cross-section | Strongly concave |
| Longitudinal axis of majority | Reflexing |
| Length of corolla tube | Short |
| Ray-floret length | 3.3 cm |
| Ray-floret width | 1.0 cm |
| Ratio length/width | Medium |
| Shape of tip | Pointed, very occasionally round |
| Disc florets | |
| Disc diameter | 0.8-1.0 cm |
| Distribution of disc florets | Numerous, clearly visible |
| | at all stages of flowering |
| Shape | Tubular |
| Color | Yellow-green 144 A |
| Receptacle shape | Conical raised |
| Reproductive Organs | |
| Stamen (present in disc florets only) | Thin 3 mm in length |
| Stamen color | Yellow-green 144 B |
| Pollen | No pollen present |
| Pollen color | Not applicable |
| Styles (present in both ray and disc | Thin |
| florets) | |
| Style color | Yellow-green 144 B |
| Style Length | 4 mm |
| Stigma color | Yellow-green 144 B |
| Stigma Width | 2 mm |
| Ovaries | Enclosed in perianth |
| Plant | |
| Form | A spray mum meant for erect |
| | culture |
| Growth habit | Upright |
| Growth rate | Very vigorous |
| Height | 125-135 cm |
| Internodes | 2.5-3.0 cm |
| Spray formation | Corymbiform |
| Stem Color | Yellow-green 147 B |
| Stem Strength | Medium |
| Stem Brittleness | Present |
| Stem Anthocyanin Coloration | Absent |
| Peduncle length | Near the top 16 cm, near the |
| | middle 23 cm, near the |
| | bottom 28 cm |
| Peduncle color | Green 147 B |
| Peduncle, attachment | Brittle |
| Peduncle, angle with stem | Small, 30° C. |
| Flowering Response(photo-periodic | 47 days |
| controlled crop, not natural season) | |
| Foliage | |
| Color | Upper side yellow-green 147 A |
| | Under side green 138 B |
| Size | large; length 12.0-14.0 cm, |
| | width 7.0-8.0 cm |
| Quantity (number per single stem) | 30-34 |
| Shape | Ovate and deeply lobed |
| Texture upper side | Fleshy and glabrous |
| Texture under side | Pubescent |
| Ribs and veins upper side | Ribs and veins well developed |
| Ribs and veins under side | Ribs and veins well developed |
| Venation arrangement | Palmate |
| Shape of the margin | Lobed |
| Shape of Base of Sinus Between | Round |
| Lateral Lobes | |
| Margin of Sinus Between Lateral | Converging |
| Lobes | |
| Shape of Base | Rounded |
| Apex | Mucronate |
| Petiole length | 2.5 cm |
| Petiole color | Green 147B |

TABLE 2
Differences with the comparison Varieties

| | | | ‘Reagan |
|---|---|---|---|
| | ‘Redock Yellow’ | ‘Redock’ | Yellow’ |
| Ray-floret color | Yellow 7B | Purple 75 C | Yellow 5C |
| Bloom height | High | High | Low |
| Cross-section of | Strongly concave | Strongly concave | Flat |
| ray-floret | | | |
| Vigour | Very vigorous | Very vigorous | Vigorous |
| Plant height | 125-135 cm | 125-135 cm | 100-125 cm |

1. A new and distinct variety of chrysanthemum plant as described and illustrated.
